(19) United States
(12) Patent Application Publication
(10) Pub. No.: US 2015/0066026 A1
(43) Pub. Date: Mar. 5, 2015

(54) SWITCH ASSEMBLIES FOR MULTI-FUNCTION, ENERGY-BASED SURGICAL INSTRUMENTS

(71) Applicant: COVIDIEN LP, Mansfield, MA (US)

(72) Inventors: Keir HART, Lafayette, CO (US); Kelley D. GOODMAN, Erie, CO (US); Diana GUNNARSON, Longmont, CO (US); Susan HIEBERT, Lyons, CO (US); Scott N. LACOSTA, Lafayette, CO (US); Weng-Kai K. LEE, Longmont, CO (US); Prakash MANLEY, Lafayette, CO (US); Charles MYERS, Louisville, CO (US); Kenneth E. NETZEL, Loveland, CO (US); Arlen J. RESCHKE, Longmont, CO (US); Paul R. ROMERO, Loveland, CO (US); Amanda K. WHITE, Port Orchard, WA (US)

(73) Assignee: COVIDIEN LP, Mansfield, MA (US)

(21) Appl. No.: 14/268,051

(22) Filed: May 2, 2014

Related U.S. Application Data
(60) Provisional application No. 61/872,001, filed on Aug. 30, 2013.

(51) Int. Cl. A61B 18/14

(52) U.S. Cl. CPC A61B 18/1445; A61B 2018/00601
USPC 606/46

(57) ABSTRACT

A surgical instrument includes an end effector assembly having jaw members movable to grasp tissue therebetween. One or both jaw members is adapted to connect to a source of energy for treating tissue grasped and one or both of the jaw members is adapted to connect to a source of energy for electrically cutting tissue. A first switch assembly is selectively activatable for supplying energy to treat tissue. A second switch is selectively activatable for supplying energy to electrically cut tissue. The tactile feel and range of motion during actuation of the second switch assembly mimics the tactile feel and range of motion of activation of a mechanical actuator that advances a cutting blade between the jaw members to mechanically cut tissue.

### CROSS REFERENCE TO RELATED APPLICATION

The present application claims the benefit of and priority to U.S. Provisional Application Ser. No. 61/872,001, filed on Aug. 30, 2013, the entire contents of which are incorporated herein by reference.

### BACKGROUND

1. Background of Related Art

The present disclosure relates to energy-based surgical instruments and, more particularly, to switch assemblies for energy-based surgical forceps configured for treating and/or cutting tissue.

2. Technical Field

A hemostat or forceps is a plier-like instrument which relies on mechanical action between its jaws to grasp, clamp, and constrict tissue. Energy-based forceps utilize both mechanical clamping action and energy, e.g., electrosurgical energy, ultrasonic energy, light energy, microwave energy, heat, etc., to affect hemostasis by heating tissue to coagulate and/or cauterize tissue. Certain surgical procedures require more than simply cauterizing tissue and rely on the unique combination of clamping pressure, precise energy control, and gap distance (i.e., distance between opposing jaw members when closed about tissue) to “seal” tissue. Typically, once tissue is sealed, the surgeon has to accurately sever the tissue along the newly formed tissue seal. Accordingly, many tissue-sealing instruments have been designed which incorporate a knife or blade member which effectively severs the tissue after forming a tissue seal. More recently, tissue-sealing instruments have been designed to allow for energy-based tissue division.

### SUMMARY

As is traditional, use of the term “distal” herein refers to an end of the apparatus or component thereof that is farther from an operator, while use of the term “proximal” herein refers to the end of the apparatus or component thereof that is closer to the operator. Further, to the extent consistent, any of the aspects and features of the present disclosure may be utilized in conjunction with any or all of the other aspects and features of the present disclosure.

In accordance with aspects of the present disclosure, a surgical instrument is provided. The surgical instrument generally includes an end effector assembly, a first switch assembly, and a second switch assembly. The end effector assembly includes first and second jaw members. One or both of the jaw members is movable relative to the other to grasp tissue therebetween. One or both of the jaw members is adapted to connect to a source of energy for treating tissue grasped between the jaw members. One or both of the jaw members is adapted to connect to a source of energy for electrically cutting tissue grasped between the jaw members. The first switch assembly is operably coupled to the end effector assembly and is selectively activatable for supplying energy to the jaw member(s) for treating tissue grasped between the jaw members. The second switch assembly is operably coupled to the end effector assembly and is selectively activatable for supplying energy to the jaw member(s) for electrically cutting tissue grasped between the jaw members. The second switch assembly is configured such that the tactile feel and range of motion during actuation of the second switch assembly to effect electrical tissue cutting mimics the tactile feel and range of motion of activation of a mechanical actuator that advances a cutting blade between the jaw members to mechanically cut tissue.

In some aspects of the present disclosure, the first switch assembly includes a depressible button.

In some aspects of the present disclosure, the first switch assembly includes a flex circuit.

In some aspects of the present disclosure, the first switch assembly includes a dome switch.

In some aspects of the present disclosure, the first and second switch assemblies are coupled to a progressive switch.

In some aspects of the present disclosure, the second switch assembly includes a rotatable lever disposed on each side of the surgical instrument. Each of the levers is rotatable from a first position to a second position to activate the second switch assembly. Further, the rotatable lever may be biased towards the first position.

In some aspects of the present disclosure, first and second shaft members are operably coupled to the end effector assembly. More specifically, the first and second shaft members are movable relative to one another between a spaced-apart position and an approximated position for moving the jaw members relative to one another to grasp tissue therebetween.

In some aspects of the present disclosure, the first switch assembly is positioned such that movement of the first and second shaft members from the spaced-apart position to the approximated position activates the first switch assembly.

In accordance with aspects of the present disclosure, a surgical instrument is provided that generally includes an end effector assembly, first and second shaft members, and a two-mode switch assembly. The end effector assembly includes first and second jaw members. One or both of the jaw members is movable relative to the other to grasp tissue therebetween. One or both of the jaw members is adapted to connect to a source of energy for treating tissue grasped between the jaw members. One or both of the jaw members is adapted to connect to a source of energy for electrically cutting tissue grasped between the jaw members. The first and second shaft members are coupled to the end effector assembly and are movable relative to one another between a spaced-apart position and first and second approximated positions for moving the jaw members relative to one another between an open position and first and second grasping positions. The first shaft member includes a flange extending therefrom towards the second shaft member. The flange includes a first portion and a second portion. The two-mode switch assembly is coupled to the second shaft member. The switch assembly includes a first switch member selectively activatable for activating the switch assembly in a first mode for supplying energy to the jaw member(s) for treating tissue grasped between the jaw members. The switch assembly further includes a second switch member selectively activatable for activating the switch assembly in a second mode for supplying energy to the jaw member(s) for electrically cutting tissue grasped between the jaw members. Movement of the shaft members to the first approximated position urges the first portion of the flange into the first switch member to activate the first switch member while movement of the shaft members to the second approximated position urges the second portion of the flange into the second switch member to activate the second switch member.

In some aspects of the present disclosure, the two-mode switch assembly is disposed within a housing positioned about the second shaft member.

In some aspects of the present disclosure, the second portion of the flange defines a relatively wide base extending from the first shaft member and the first portion of the flange defines a relatively narrow extension extending from the base.

In some aspects of the present disclosure, the first switch member of the two-mode switch assembly is disposed within an aperture defined through the second switch member.

In some aspects of the present disclosure, a safety selector is provided. The safety selector is selectively movable between a first position, inhibiting activation of both the first and second switch members of the two-mode switch assembly, a second position inhibiting activation of the second switch member of the two-mode switch assembly but permitting activation of the first switch member of the two-mode switch assembly, and a third position permitting activation of both the first and second switch members of the two-mode switch assembly.

In some aspects of the present disclosure, the safety selector includes one or more gripping flanges The gripping flange(s) is configured to facilitate movement of the safety selector between the first, second, and third positions.

In some aspects of the present disclosure, the safety selector is slidable along the second shaft member and relative to the two-mode switch assembly between the first, second, and third positions.

In accordance with aspects of the present disclosure, a surgical instrument is provided generally including an end effector assembly, a first switch member, a second switch member, and an activation member. The end effector assembly includes first and second jaw members. One or both of the jaw members is movable relative to the other to grasp tissue therebetween. One or both of the jaw members is adapted to connect to a source of energy for treating tissue grasped between the jaw members. One or both of the jaw members is adapted to connect to a source of energy for electrically cutting tissue grasped between the jaw members. The first switch member is selectively activatable for supplying energy to the jaw member(s) for treating tissue grasped between the jaw members. The second switch member is selectively activatable for supplying energy to the jaw member(s) for electrically cutting tissue grasped between the jaw members. The activation member includes first and second activation components. The activation member is movable in a first direction for urging the first activation component into the first switch member for activating the first switch member and is movable in a second direction opposite the first direction for urging the second activation component into the second switch member for activating the second switch member.

In some aspects of the present disclosure, the activation member includes a rotating assembly having first and second flanges, the rotating assembly is rotatable in the first direction such that the first flange is urged into contact with the first switch member to activate the first switch member and rotatable in the second direction such that the second flange is urged into contact with the second switch member to activate the second switch member.

In some aspects of the present disclosure, the rotating assembly is biased towards a neutral position wherein both the first and second flanges are displaced from the first and second switch members, respectively.

In some aspects of the present disclosure, the activation member includes a lever disposed about a fulcrum. The lever includes a first end and a second end and is tiltable about the fulcrum. In particular, the lever is tiltable about the fulcrum in the first direction such that the first end is urged into contact with the first switch member to activate the first switch member and tiltable in the second direction such that the second end is urged into contact with the second switch member to activate the second switch member.

### BRIEF DESCRIPTION OF THE DRAWINGS

Various aspects and features of the present disclosure are described herein with reference to the drawings wherein:

FIG. 1 is a perspective view of a surgical forceps provided in accordance with the present disclosure;

FIG. 2 is an enlarged, perspective view of a distal end of the forceps of FIG. 1;

FIG. 3 is a perspective view of the forceps of FIG. 1 with parts separated;

FIG. 4A is a longitudinal, cross-sectional view of one configuration of switch assemblies for use with a forceps similar to the forceps of FIG. 1;

FIG. 4B is an enlarged view of the area of detail indicated as “4B” in FIG. 4A;

FIG. 4C is a side, cross-sectional view of the first switch assembly of FIG. 4B with the outer portion of the first switch assembly removed;

FIG. 5A is a perspective view of the first switch assembly of FIG. 4A;

FIG. 5B is a perspective view of the first switch assembly of FIG. 4A with the outer portion of the first switch assembly removed;

FIG. 5C is a top view of the first switch assembly of FIG. 4A;

FIG. 6A is a longitudinal, cross-sectional view of another configuration of switch assemblies for use with a forceps similar to the forceps of FIG. 1;

FIG. 6B is an enlarged view of the area of detail indicated as “6B” in FIG. 6A;

FIG. 6C is a cross-sectional view of the second switch assembly of FIG. 6A;

FIG. 7A is a longitudinal, cross-sectional view of another configuration of switch assemblies for use with a forceps similar to the forceps of FIG. 1;

FIG. 7B is an enlarged view of the area of detail indicated as “7B” in FIG. 7A;

FIG. 8A is a longitudinal, cross-sectional view of another configuration of switch assemblies for use with a forceps similar to the forceps of FIG. 1;

FIG. 8B is an enlarged view of the proximal end of the forceps of FIG. 8A;

FIG. 8C is a top view of the switch assemblies of FIG. 8A;

FIG. 8D is a top view of the switch assemblies of FIG. 8A with a safety mechanism disposed in a first condition;

FIG. 8E is a top view of the switch assemblies of FIG. 8A with the safety mechanism disposed in a second condition;

FIG. 8F is a top view of the switch assemblies of FIG. 8A with the safety mechanism disposed in a third condition;

FIG. 9A is a side view of another configuration of switch assemblies for use with a forceps similar to the forceps of FIG. 1;

FIG. 9B is an enlarged, partial cross-sectional view of the first and second switch assemblies of FIG. 9A;

FIG. 10A is a longitudinal, cross-sectional view of another configuration of switch assemblies for use with a forceps similar to the forceps of FIG. 1;

FIG. 10B is an enlarged view of the area of detail indicated as “10B” in FIG. 10A; and

FIG. 10C is a cross-sectional view of the switch assemblies of FIG. 10B with the outer portions of the switch assemblies removed.

### DETAILED DESCRIPTION

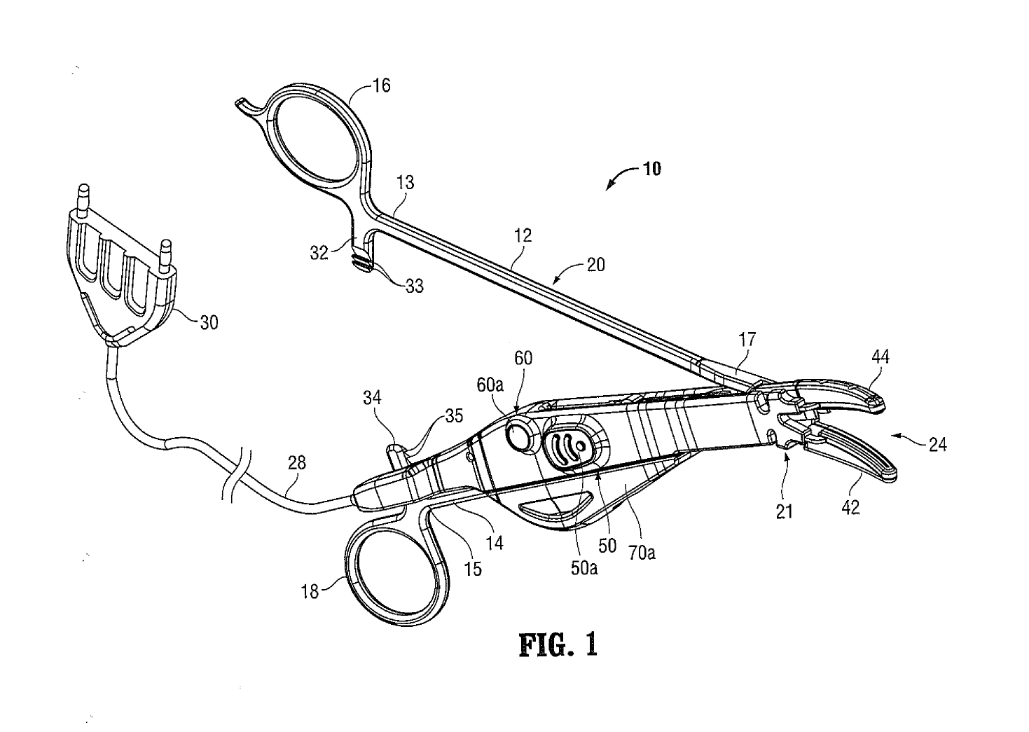

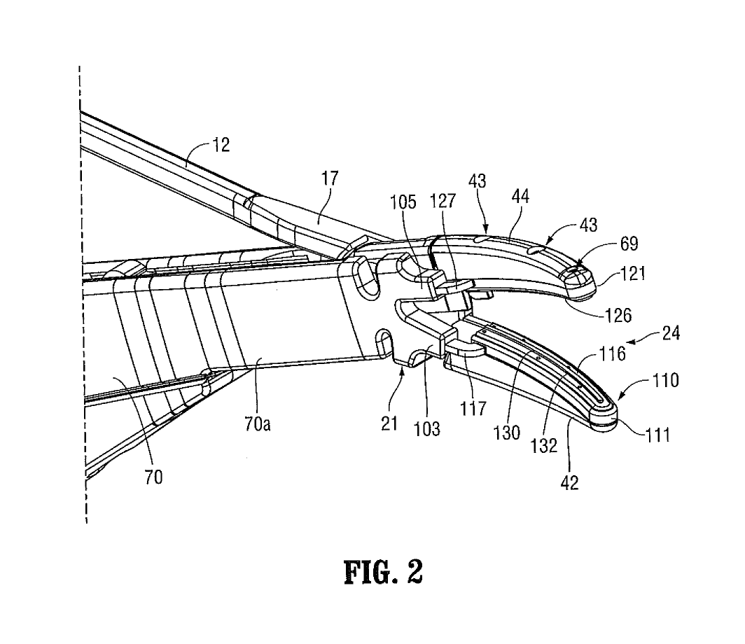

Referring now to FIGS. 1-3, a forceps provided in accordance with the present disclosure is shown generally identified by reference numeral 10. Forceps 10 is configured for grasping, electrically treating, and electrically (including electro-mechanically) dissecting tissue. As such, and as will be described in greater detail below, forceps 10 includes multiple switch assemblies 50, 60 configured to facilitate electrical activation of forceps 10 in various modes of operation, e.g., an electrical treatment mode and an electrical cutting mode. Various embodiments of switch assemblies are shown in FIGS. 1-10C and described herein, any or all of which may be used in conjunction with any or all of the other switch assemblies provided in accordance with the present disclosure, depending on a particular purpose. Although the various switch assemblies are shown and configured for use with forceps similar to forceps 10, it is contemplated that the various switch assemblies, aspects, and features of the present disclosure are equally applicable for use with any suitable multi-function surgical instrument. Obviously, different connections and considerations apply to each particular instrument and the assemblies and/or components thereof; however, the aspects, features, and operating characteristics of the switch assemblies remain generally consistent regardless of the particular instrument, assemblies, and/or components provided. For the purposes herein, forceps 10 is generally described.

Continuing with reference to FIGS. 1-3, forceps 10, shown configured for use in open and/or laparoscopic surgical procedures, generally includes a mechanical forceps 20 and a disposable portion that includes a housing 70 and an electrode assembly 21, both of which are releasably engagable with mechanical forceps 20. Mechanical forceps 20 includes first and second elongated shaft members 12, 14. Elongated shaft member 12 includes proximal and distal end portions 13, 17, respectively, and elongated shaft member 14 includes proximal and distal end portions 15, 19, respectively. Disposed at proximal end portions 13, 15 of shaft members 12, 14 are handle members 16, 18, respectively, that are configured to allow a user to effect movement of at least one of shaft members 12, 14 relative to the other. Distal end portions 17, 19 of mechanical forceps 20 cooperate to define an end effector assembly 24 having opposing jaw members 42, 44 that extend distally from respective shaft members 12, 14. Jaw members 42, 44 are movable relative to each other in response to movement of shaft members 12, 14.

Shaft members 12, 14 are coupled to one another towards distal end portions 17, 19, respectively, thereof via a pivot 25 such that movement of shaft members 12, 14 relative to one another from a spaced-apart position to one or more approximated positions effects corresponding movement of jaw members relative to one another from an open configuration, wherein jaw members 42, 44 are disposed in spaced relation relative to one another, to one or more closed positions, wherein jaw members 42, 44 cooperate to grasp tissue therebetween.

Each shaft member 12, 14 further includes a ratchet portion 32, 34, respectively. Each ratchet portion 32, 34 extends from the proximal end portion 13, 15 of its respective shaft member 12, 14 towards the other ratchet 32, 34 in a generally vertically aligned manner such that the inner facing surfaces of each ratchet 32, 34 abut one another when shaft members 12, 14 are approximated. Each ratchet 32, 34 includes a plurality of flanges 33, 35, respectively, that project from the inner facing surface of each ratchet 32, 34 such that ratchets 32, 34 may interlock at one or more positions corresponding to one or more closed positions of jaw members 42, 44. These one or more closed positions of jaw members 42, 44 each impart a specific closure pressure to tissue grasped between jaw members 42, 44 of end effector assembly 24, thus allowing for effective treatment of a wide range of tissue types and sizes.

Referring still to FIGS. 1-3, housing 70 includes a pair of housing halves 70a, 70b configured to matingly engage and releasably encompass at least a portion of shaft member 14 therebetween. An interior of each housing half 70a, 70b may include a plurality of cooperating mechanical interfaces, e.g., protrusions and recesses, pins and apertures, or other suitable latching mechanisms, disposed at various positions to effect mechanical coupling of housing halves 70a, 70b to form housing 70 about shaft member 14.

Disposable electrode assembly 21 extends distally from housing 70 and is bifurcated at the distal end thereof to define two portions 103 and 105. First portion 103 is configured to releasably engage jaw member 42 and support a first electrode 110, while second portion 105 is configured to releasably engage jaw member 44 and support a second electrode 120, as will be described in greater detail below. A pair of wires 61, 62 are electrically connected to the electrodes 110, 120, respectively, extend through housing 70, couple to switch assemblies 50, 60, and ultimately bundle to form a cable 28 that terminates at a terminal connector 30. Terminal connector 30 is configured to releasably couple to a suitable energy source such as an electrosurgical generator (not shown) for providing energy to forceps 10.

Electrode 110 includes an electrically conductive sealing surface 116 configured to conduct electrosurgical energy therethrough, while an electrically insulative substrate 111 of first portion 103 serves to electrically insulate jaw member 42 from sealing surface 116. Sealing surface 116 and substrate 111 are attached to one another by any suitable method of assembly such as, for example, snap-fit engagement or by overmolding substrate 111 to sealing surface 116. Substrate 111 includes a plurality of bifurcated anchor members 112 extending therefrom that are configured to compress during insertion into a corresponding plurality of sockets 41 disposed at least partially through an inner facing surface 45 of jaw member 42 and subsequently expand to releasably engage corresponding sockets 41 after insertion to couple first portion 103 to inner facing surface 45 of jaw member 42. Substrate 111 also includes an alignment pin (not shown, similar to pin 124) that is configured to engage an aperture 67 disposed at least partially through inner facing surface 45 of jaw member 42 to ensure proper alignment of electrode 110 with jaw member 42 during assembly. Sealing surface 116 includes a proximal extension portion 117 configured to couple to a first prong member 118 of disposable electrode assembly 21 to thereby electrically connect sealing surface 116 to wire 61.

With continued reference to FIGS. 1-3, and similarly as described above with respect to first portion 103, second portion 105 includes an electrode 120 having an electrically conductive sealing surface 126 configured to conduct electrosurgical energy therethrough. Second portion 105 further includes an electrically insulative substrate 121 including a plurality of bifurcated anchor members 122 extending therefrom that are configured to compress during insertion into a corresponding plurality of sockets 43 disposed at least partially through an inner facing surface 47 of jaw member 44 and subsequently expand to releasably engage corresponding sockets 43 after insertion to couple second portion 105 to inner facing surface 47 of jaw member 44. Substrate 121 also includes an alignment pin 124 that is configured to engage an aperture 69 disposed at least partially through inner facing surface 47 of jaw member 44 to ensure proper alignment of electrode 120 with jaw member 44 during assembly. Sealing surface 126 includes a proximal extension portion 127 configured to mechanically couple to a second prong (not shown, similar to first prong 118) of disposable electrode assembly 21 to thereby electrically connect sealing surface 126 to wire 62.

One of the first and second portions 103, 105 of disposable electrode assembly 21, e.g., first portion 103, further includes an electrical cutting electrode 130 disposed within a longitudinal slot 132 extending along sealing surface 116. A portion of substrate 111 disposed within slot 132 extends between electrical cutting electrode 130 and sealing surface 116 on either side of electrical cutting electrode 130 to electrically insulate electrical cutting electrode 130 from sealing surface 126. Substrate 111 further extends between electrical cutting electrode 130 and jaw member 44 to electrically insulate electrical cutting electrode 130 from jaw member 42. The other portion, e.g., second portion 105, likewise includes a slot (not shown, similar to slot 132) defined within the sealing surface 126. A portion of substrate 121 is disposed within the slot (not shown) to oppose cutting electrode 130, thus maintaining electrical insulation between electrical cutting electrode 130 and both sealing surface 126 and jaw member 44 when jaw members 42, 44 are disposed in the one or more closed positions.

A third prong 138 of disposable electrode assembly 21 coupled to a third wire 63 is engaged to electrical cutting electrode 130 to electrically connect electrical cutting electrode 130 to third wire 63. Third wire 63 extends through housing 70, couples to first and second switch assemblies 50, 60, and ultimately bundles with first and second wires 61, 62 to form cable 28.

Continuing with reference to FIGS. 1-3, to selectively control the supply of energy to electrodes 110, 120, 130 for treating and/or dissecting tissue grasped between jaw members 42, 44, housing 70 includes first and second switch assemblies 50, 60, each including a pair of depressible activation buttons 50a, 50b and 60a, 60b, respectively. First switch assembly 50 includes a depressible activation button 50a, 50b disposed on either side of housing 70. Activation buttons 50a, 50b of first switch assembly 50 are electrically coupled between the energy source (not shown) and first and second electrodes 110, 120 via wires 61, 62. As such, depression of either or both of activation buttons 50a, 50b energizes electrode 110 to a relative positive potential and electrode 120 to a relatively negative potential (although this configuration may be reversed) to establish a potential gradient between electrodes 110, 120 for conducting energy through tissue grasped between jaw members 42, 44 to treat, e.g., seal, tissue. Second switch assembly 60 includes a depressible activation button 60a, 60b disposed on either side of housing 70. Activation buttons 60a, 60b of second switch assembly 60 are coupled between the energy source (not shown) and electrical cutting electrode 130 via wire 63 such that depression of either or both of activation buttons 60a, 60b energizes electrical cutting electrode 130 to a relatively positive potential and electrodes 110, 120 to a relatively negative potential. Accordingly, a potential gradient is established therebetween for conducting energy through tissue grasped between jaw members 42, 44 to cut tissue.

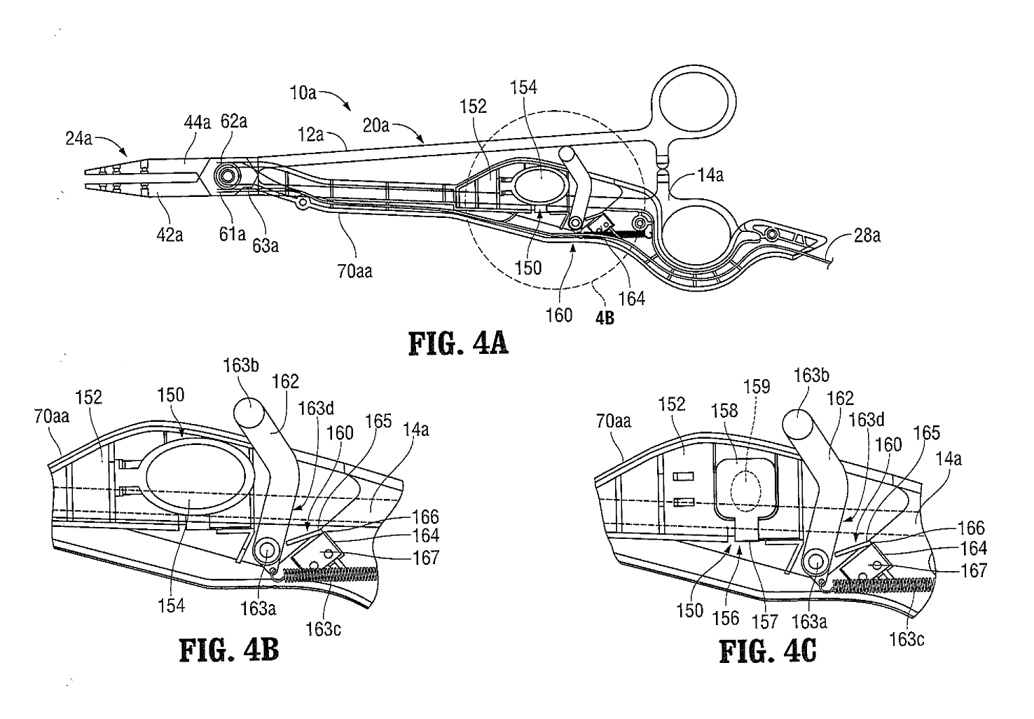

Turning now to FIGS. 4A-10C, various embodiments of switch assemblies configured for use with forceps 10 (or forceps similar to forceps 10) for selectively controlling the supply of energy to electrodes 110, 120, 130 (or electrodes similar to electrodes 110, 120, 130) for treating and/or dissecting tissue grasped between jaw members 42, 44 (or jaw members similar to jaw members 42, 44) are described. For the purpose of brevity, and given the description of forceps 10 above, only the distinguishing aspects and features of the switch assemblies of FIGS. 4A-10C and the forceps used therewith will be described in detail below.

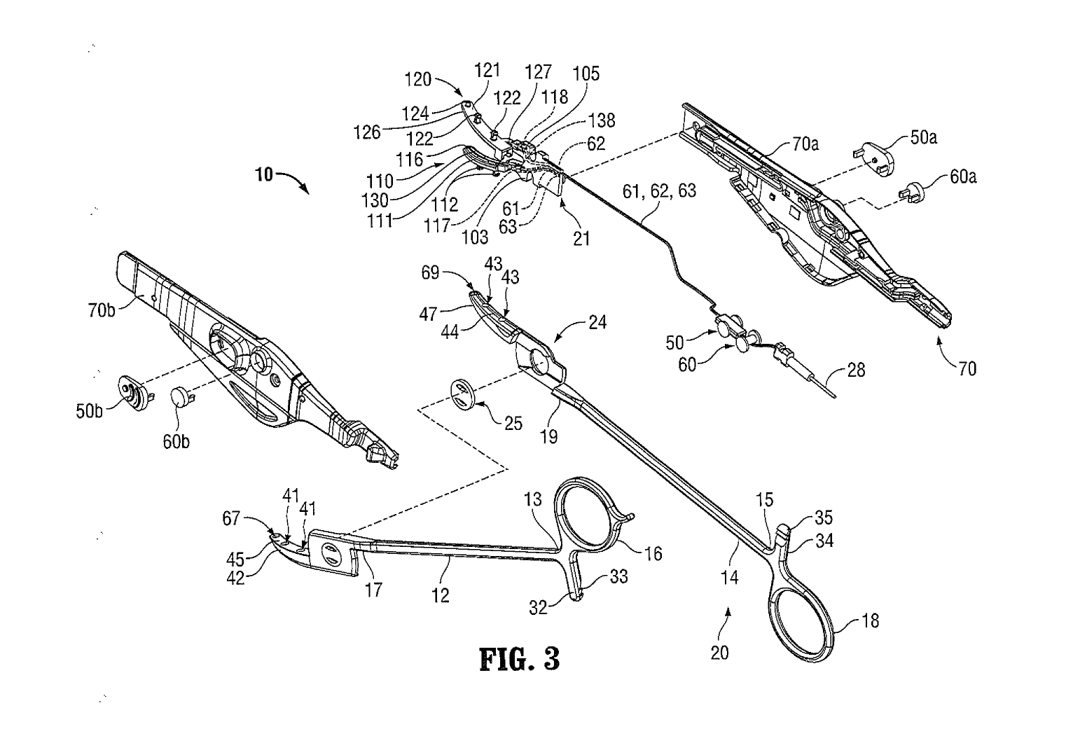

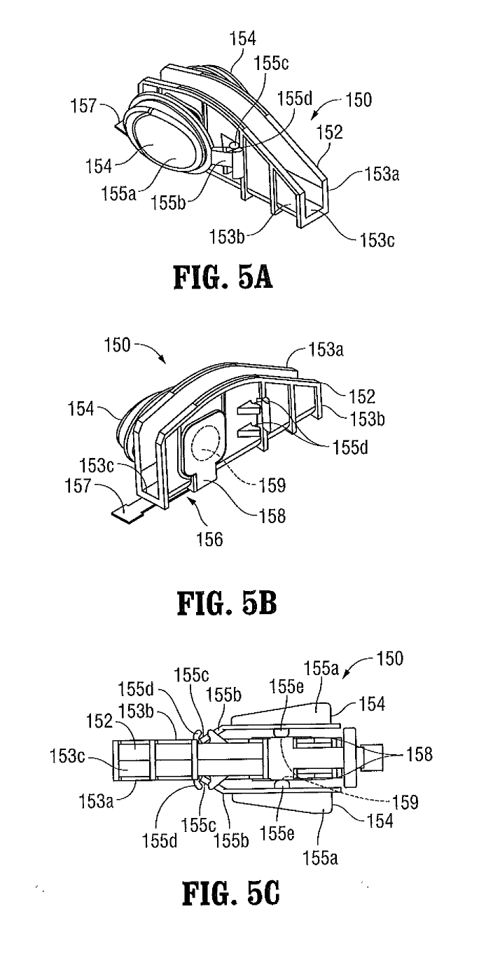

With reference to FIGS. 4A-4C and 5A-5C, another forceps 10a similar to forceps 10 (FIGS. 1-3) is shown including first and second switch assemblies 150, 160 disposed within housing 70aa and coupled between the source of energy (not shown) and end effector assembly 24a via wires 61a, 62a, 63a. With particular reference to FIGS. 5A-5C, first switch assembly 150 is configured for selectively energizing electrodes, e.g., electrodes similar to electrodes 110, 120 (FIG. 3), for operation in a tissue treatment mode, e.g., for sealing tissue grasped between jaw members 42a, 44a. First switch assembly 150 includes a frame 152, a pair of depressible activation members 154 and a flex circuit assembly 156. Frame 152 defines first and second spaced-apart walls 153a, 153b, respectively, each of which is configured to operably receive one of the depressible activation members 154 on an outwardly-facing side thereof. A slot 153c defined between first and second walls 153a, 153b is configured to receive shaft member 14a of mechanical forceps 20a for releasably engaging first switch assembly 150 about mechanical forceps 20a. Frame 152 may be fixedly engaged to one of the housing portions of housing 70aa or may be releasably engagable with one of the housing portions of housing 70aa upon engagement of housing 70aa about shaft member 14a of mechanical forceps 20a. In either configuration, shaft member 14a is inserted through the open end of slot 153c of frame 152 until shaft member 14a is seated between walls 153a, 153b at the base of slot 153c of frame 152. Openings in the housing portions of housing 70aa adjacent depressible activation members 154 provide user-access to depressible activation members 154 from either side of housing 70aa to permit selective activation of first switch assembly 150.

Continuing with reference to FIGS. 5A-5C, and as mentioned above, a depressible activation member 154 is operably engaged to each wall 153a, 153b of frame 152. More specifically, depressible activation members 154 each include a finger-contact portion 155a having a connector 155b extending therefrom. Finger-contact portions 155a provide an expanded surface area configured to facilitate a user's ability to depress depressible activation members 154. Connectors 155b extend from finger-contact portions 155a and define transverse pivot bars 155c at the free ends thereof. Transverse pivot bars 155c are received within hinge recesses 155d defined on the outwardly-facing surfaces of walls 153a, 153b of frame 152. This hinged-engagement of connectors 155b of depressible activation members 154 to frame 152 allows finger-contact portions 155a of depressible activation members 154 to be selectively depressible from an initial position to a depressed position for selectively activating first switch assembly 150, as will be described in greater detail below. Protrusions 155e (FIG. 5C) defined on the inwardly-facing surfaces of finger-contact portions 155a of depressible activation members 154 facilitate the activation of first switch assembly 150. Further, connectors 155b, pivot bars 155c, and/or hinge recesses 155d may be configured such that finger-contact portions 155a are biased towards the initial position. Alternatively or additionally, a biasing member (not shown) may be provided for similar purposes.

Flex circuit assembly 156 of first switch assembly 150 includes a body 157 extending along the base of frame 152 and a pair of flanges 158 that extend along walls 153a, 153b of frame 152 adjacent finger-contact portions 155a of depressible activation members 154. Flanges 158 each include a dome switch 159 disposed on an outwardly facing surface thereof. Dome switches 159 are electrically coupled to wires 61a, 62a via the internal circuitry of flex circuit assembly 156 so as to selectively permit the transmission of energy from the energy source (not shown) to the electrodes, e.g., electrodes 110, 120 (FIG. 3). Dome switches 159 are normally biased towards an un-actuated state, disconnecting the electrical path through first switch assembly 150. However, upon actuation of one or both of dome switches 159, e.g., via depression of one or both depressible activation members 154 such that at least one of protrusions 155e urges at least one of dome switches 159 to an actuated position, the electrical path is reestablished, thus allowing energy transmission along wires 61a, 62a from the energy source (not shown), through first switch assembly 150, to the electrodes, e.g., electrodes 110, 120 (FIG. 3). More specifically, upon activation of one or both of dome switches 159, electrode 110 (FIG. 3) is energized to a relative positive potential and electrode 120 (FIG. 3) to a relatively negative potential (although this configuration may be reversed) to establish a potential gradient between electrodes 110, 120 (FIG. 3) for conducting energy through tissue grasped between jaw members 42a, 44a to treat, e.g., seal, tissue.

Referring again to FIGS. 4A-4C, second switch assembly 160 is provided to selectively control the supply of energy to the electrodes, e.g., electrodes 110, 120, 130 (FIG. 3), for operation of end effector assembly 24a in an electrical cutting mode, e.g., to electrically cut tissue grasped between jaw members 42a, 44a. Second switch assembly 160 includes a pair of activation levers 162 disposed on either side of shaft member 14a and a pair contact switch members 164 disposed on either side of shaft member 14a (although only one of each is shown in FIGS. 4A-4C). Each activation lever 162 is pivotably coupled to one of the housing portions of housing 70aa via a pivot 163a at a first end thereof and extends from housing 70aa, toward shaft member 12a, to a free end thereof. A transverse, outwardly-protruding nub 163b is disposed at the free end of each lever 162 to facilitate grasping either lever 162 for rotating either lever 162 about its respective pivot 163a. As will be described in greater below, levers 162 are pivotable about pivots 163a and relative to housing 70aa from a distal position to a proximal position to energize electrodes 110, 120, 130 (FIG. 3) for conducting energy through tissue grasped between jaw members 42a, 44a to cut tissue. Further, a biasing member 163c may be provided to bias lever 162 towards the distal position.

Contact switch members 164 are electrically coupled to wires 61a, 62a, 63a via the internal circuitry of contact switch members 164 so as to selectively permit the transmission of energy from the energy source (not shown) to electrodes 110, 120, 130 (FIG. 3). More specifically, contact switch members 164 each include a contact finger 165 that is normally biased, e.g., via a living hinge 166, towards an un-actuated state, disconnecting the electrical path through second switch assembly 160. However, upon urging of either of contact fingers 165 into contact with the respective body 167 of the contact switch member 164, the electrical path is reestablished, thus allowing energy transmission along wires 61a, 62a, 63a from the energy source (not shown) through second switch assembly 160 to end effector assembly 24a to energize electrical cutting electrode 130 (FIG. 3) to a relatively positive potential and electrodes 110, 120 (FIG. 3) to a relatively negative potential for conducting energy through tissue grasped between jaw members 42a, 44a to cut tissue. Contact switch members 164 may be configured as on/off switches, e.g., wherein electrical contact between contact fingers 165 and body 167 supplies constant energy to end effector assembly 24a, or, alternatively, may be configured as progressive switches, e.g., where the further contact fingers 165 are urged into contact with bodies 167, the more energy is supplied to end effector assembly 24a.

As mentioned above, levers 162 are pivotable about pivots 163a and relative to housing 70aa to activate contact switch members 164 to thereby energize electrodes 110, 120, 130 (FIG. 3). By providing a lever 162 and corresponding contact switch member 164 on either side of forceps 10a, activation of forceps 10a in the electrical cutting mode can be effected from either side of forceps 10, e.g., via actuating either of levers 162, depending on the surgeon's preference, anatomical considerations, or other factors. With levers 162 disposed in the distal position, proximally-facing surfaces 163d of levers 162 are spaced-apart from contact fingers 165 of contact switch members 164 such that contact fingers 165 remain biased towards the un-actuated state. Accordingly, in the distal position of levers 162, the electrical path through second switch assembly 160 is disconnected. Upon pivoting of either of levers 162 from the distal position towards the proximal position, the proximally-facing surface 163d of the respective lever 162 contacts the corresponding contact finger 165 and urges the contact finger 165 into contact with the respective body 167 of the contact switch member 164 to reestablish the electrical path through second switch assembly 160 and thereby transmit energy from the energy source (not shown) to end effector assembly 24a for operation in the electrical cutting mode.

In embodiments where contact switch member 164 is configured as an on/off switch, the user may pivot lever 162 to the proximal position and maintain lever 162 in the proximal position sufficiently long so as to effect tissue cutting. The energy source (not shown), for example, may provide an audible alert indicating completion of tissue cutting, although other indicators are also contemplated. Alternatively, full pivoting of lever 162 from the distal position to the proximal position, which is slowed by the bias of biasing member 163c and living hinge 166, provides sufficient “ON” time to electrically cut tissue grasped between jaw members 42a, 44a. Thus, the surgeon is provided with a similar tactile feel and range of motion for electrically cutting tissue as compared to the more traditional approach of mechanically advancing a blade (not shown) between jaw members 42a, 44a to mechanically cut tissue grasped therebetween. In other words, activation of second switch assembly 160 mimics the activation of a mechanical blade (not shown). Further, by pivoting lever 162 through its full range of motion in this manner, energy-based tissue cutting can be achieved without the need for other indicators of cutting completion (although such indicators may also be provided).

In embodiments where contact switch member 164 is configured as a progressive switch, full pivoting of lever 162 from the distal position to the proximal position incrementally or continuously increases the energy applied to end effector assembly 24a, e.g., in accordance with a pre-determined electrical cutting energy supply profile, such that, similarly as above, pivoting lever 162 through its full range of motion effects energy-based tissue cutting using the same tactile feel and range of motion as used in advancing a mechanical blade (not shown), e.g., mimicking mechanical tissue cutting.

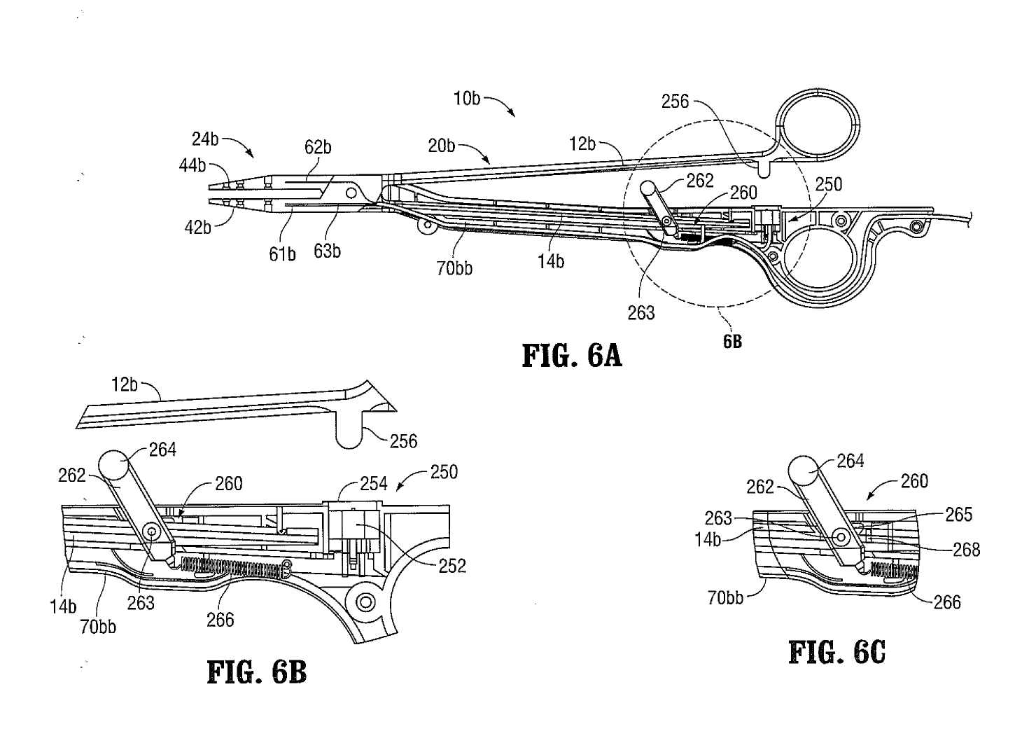

Turning now to FIGS. 6A-6C, another forceps 10b similar to forceps 10 (FIGS. 1-3) is shown including first and second switch assemblies 250, 260 disposed within housing 70bb and coupled between the source of energy (not shown) and end effector assembly 24b via wires 61b, 62b, 63b. Similarly as described above with respect to first switch assembly 150 (FIGS. 5A-5C), first switch assembly 250 is configured for selectively energizing electrodes, e.g., electrodes 110, 120 (FIG. 3), for operation in a tissue treatment mode, e.g., for sealing tissue grasped between jaw members 42b, 44b.

First switch assembly 250 includes an outer sleeve 252 and an inner activation button 254. Outer sleeve 252 is fixedly disposed within housing 70bb, while inner activation button 254 is slidably positioned within outer sleeve 252 and extends from outer sleeve 252 and housing 70bb towards shaft member 12b. Inner activation button 254 is biased towards an un-activated position, wherein activation button 254 extends further towards shaft member 12b. Shaft member 12b of mechanical forceps 20b includes an activation flange 256 extending towards shaft member 14b and, in particular, towards activation button 254 such that, upon sufficient approximation of shaft members 12b, 14b, activation flange 256 contacts activation button 254 and urges activation button 254 inwardly into outer sleeve 252 to activate first switch assembly 250. Upon activation of first switch assembly 250, energy is transmitted along wires 61b, 62b from the energy source (not shown), through first switch assembly 250, to the electrodes, e.g., electrodes 110, 120, respectively (FIG. 3). More specifically, upon activation of first switch assembly 250, electrode 110 (FIG. 3) is energized to a relative positive potential and electrode 120 (FIG. 3) to a relatively negative potential (although this configuration may be reversed) to establish a potential gradient between electrodes 110, 120 (FIG. 3) for conducting energy through tissue grasped between jaw members 42b, 44b to treat, e.g., seal, tissue.

Continuing with reference to FIGS. 6A-6C, second switch assembly 260 is provided to selectively control the supply of energy to the electrodes, e.g., electrodes 110, 120, 130 (FIG. 3), for operation of end effector assembly 24b in an electrical cutting mode, e.g., to electrically cut tissue grasped between jaw members 42b, 44b. Second switch assembly 260 includes a pair of activation levers 262 disposed on either side of shaft member 14b and a pair activation buttons 268 disposed on either side of shaft member 14b proximally adjacent respective levers 262 (although only one of each is shown in FIGS. 6A-6C).

Each activation lever 262, as best shown in FIGS. 6B and 60, is pivotably coupled to one of the housing portions of housing 70bb via a pivot 263 at a first end thereof and extends from housing 70bb, toward shaft member 12b, to a free end thereof. A transverse, outwardly-protruding nub 264 is disposed at the free end of each lever 262 to facilitate grasping either lever 262 for rotating either lever 262 about its respective pivot 263a. Each activation lever 262 further includes a protrusion member 265 extending proximally therefrom. As will be described in greater detail below, protrusion members 265 are configured to contact activation buttons 268 to activate second switch assembly 260 upon rotation of one or both of activation levers 262 from a distal position to a proximal position. A biasing member 266 is also provided to bias lever 262 towards the distal position.

Activation buttons 268 of second switch assembly 260 are electrically coupled to wires 61b, 62b, 63b to selectively permit the transmission of energy from the energy source (not shown) to the electrodes, e.g., electrodes 110, 120, 130 (FIG. 3). As best shown in FIG. 6C, activation buttons 268 are normally biased towards an un-actuated state, disconnecting the electrical path through second switch assembly 260. However, upon urging of either of protrusion members 265 of levers 262 into contact with the respective activation button 268, the electrical path is reestablished, thus allowing energy transmission along wires 61b, 62b, 63b from the energy source (not shown) through second switch assembly 260 to end effector assembly 24b to energize electrical cutting electrode 130 to a relatively positive potential and electrodes 110, 120 (FIG. 3) to a relatively negative potential for conducting energy through tissue grasped between jaw members 42b, 44b to cut tissue.

Similarly as described above with respect to second switch assembly 160 (FIGS. 4A-5C), second switch assembly 260 may be configured as an on/off switch or, alternatively, may be configured as a progressive switch. In either configuration, activation of second switch assembly 260 effects energy-based tissue cutting that mimics the tactile feel and range of motion used in actuating a mechanical actuator for mechanical tissue cutting.

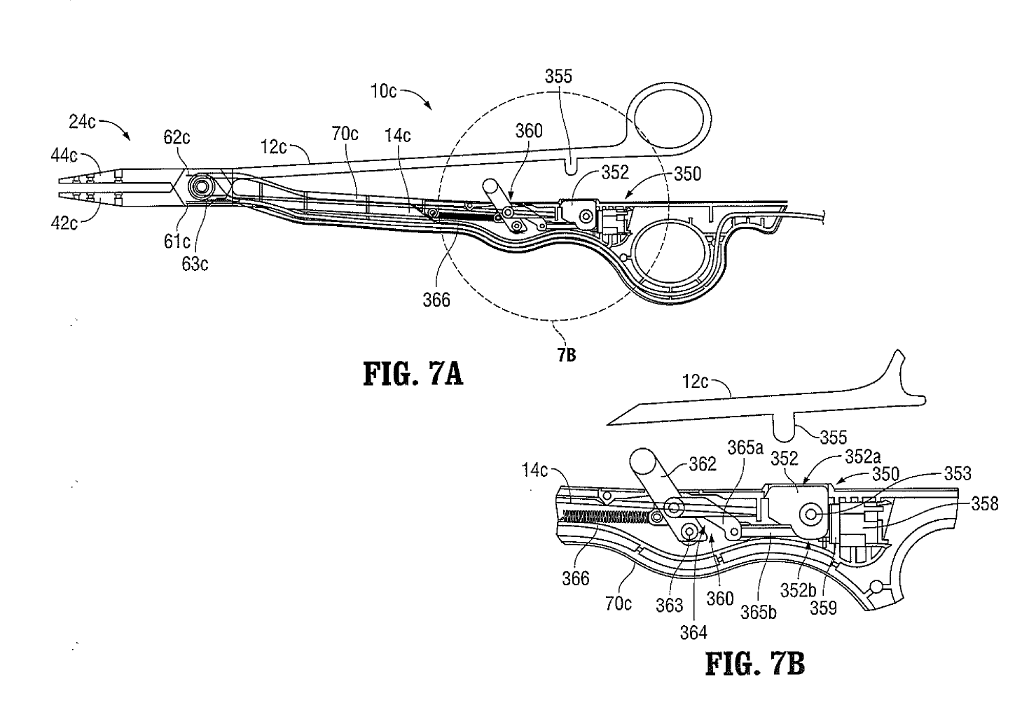

Turning now to FIGS. 7A-7B, another forceps 10c similar to forceps 10 (FIGS. 1-3) is shown including first and second switch assemblies 350, 360 disposed within housing 70c and coupled between the source of energy (not shown) and end effector assembly 24c via wires 61c, 62c, 63c. Similarly as described above with respect to the previous embodiments, first switch assembly 350 is configured for selectively energizing electrodes, e.g., electrodes 110, 120 (FIG. 3), for operation in a tissue treatment mode, e.g., for sealing tissue grasped between jaw members 42c, 44c.

First switch assembly 350 includes a rocker 352 operably positioned relative to a two-stage activation switch 358. A pivot pin 353 pivotably retains rocker 352 within a recess defined within housing 70c. Rocker 352 is pivotable about pivot pin 353 between an un-actuated position and an actuated position for activating forceps 10c for operation in a tissue treatment mode. More specifically, rocker 352 defines an exposed contact surface 352a that is positioned to oppose activation flange 355 of shaft member 12c and a protruding activation surface 352b that is configured to selectively contact and activate two-stage activation switch 358 in the first stage, or mode, e.g., the tissue treatment mode.

Activation flange 355 of shaft member 12c is offset relative to pivot pin 353 such that, upon sufficient approximation of shaft members 12c, 14c, activation flange 355 contacts exposed contact surface 352a of rocker 352 and urges rocker 352 to rotate about pivot pin 353, thereby rotating protruding activation surface 352b of rocker 352 into two-stage activation switch 358 to depress activation button 359 a first amount corresponding to the first stage, or mode of two-stage activation switch 358. With two-stage activation switch 358 activated in this first stage, or mode, energy is transmitted along wires 61c, 62c from the energy source (not shown), through first switch assembly 350, to the electrodes, e.g., electrodes 110, 120 (FIG. 3), such that electrode 110 (FIG. 3) is energized to a relative positive potential and electrode 120 (FIG. 3) to a relatively negative potential (although this configuration may be reversed) to establish a potential gradient between electrodes 110, 120 (FIG. 3) for conducting energy through tissue grasped between jaw members 42c, 44c to treat, e.g., seal, tissue,

Continuing with reference to FIGS. 7A and 7B, second switch assembly 360 is provided to selectively control the supply of energy to the electrodes, e.g., electrodes 110, 120, 130 (FIG. 3), for operation of end effector assembly 24c in an electrical cutting mode, e.g., to electrically cut tissue grasped between jaw members 42c, 44c. Second switch assembly 360 includes a pair of activation levers 362 disposed on either side of shaft member 14c, each of which are coupled to a linkage assembly 364 that is operably positioned relative to two-stage activation switch 358 such that, upon actuation of either activation lever 362, two-stage activation switch 358 is activated in the second stage, or mode, wherein energy is supplied to end effector assembly 24c for electrically cutting tissue.

Each activation lever 362 is pivotably coupled to one of the housing portions of housing 70c via a pivot 363 at a first end thereof and extends from housing 70c, toward shaft member 12c, to a free end thereof. A transverse, outwardly-protruding nub 364 is disposed at the free end of each lever 362 to facilitate grasping and pivoting the lever 362 about pivot 363. A biasing member 366 is also provided to bias lever 362 towards a distal position.

As mentioned above, each activation lever 362 is coupled to a linkage assembly 364. More specifically, a first linkage bar 365a is pivotably coupled to and extends proximally from an intermediate portion of each activation lever 362, e.g., between the first and free ends thereof, while a second linkage bar 365b is pivotably coupled to and extends proximally from each first linkage bar 365a. Second linkage bars 365b each define a free end that is configured to selectively contact and depress activation button 359 of two-stage activation switch 358 a second amount corresponding to the second stage, or mode of two-stage activation switch 358 upon pivoting of the corresponding lever 362 about its pivot 363 from the distal position to a proximal position. Activation of activation button 359 of second switch assembly 360 in the second stage establishes and electrical path such that energy is transmitted along wires 61c, 62c, 63c from the energy source (not shown), through second switch assembly 360 to end effector assembly 24c to energize electrical cutting electrode 130 to a relatively positive potential and electrodes 110, 120 (FIG. 3) to a relatively negative potential for conducting energy through tissue grasped between jaw members 42c, 44c to cut tissue.

Similarly as described above with respect to second switch assembly 160 (FIGS. 4A-5C), second switch assembly 360 may be configured as an on/off switch or, alternatively, may be configured as a progressive switch. In either configuration, activation of second switch assembly 360 effects energy-based tissue cutting that mimics the tactile feel and range of motion used in actuating a mechanical actuator for mechanical tissue cutting.

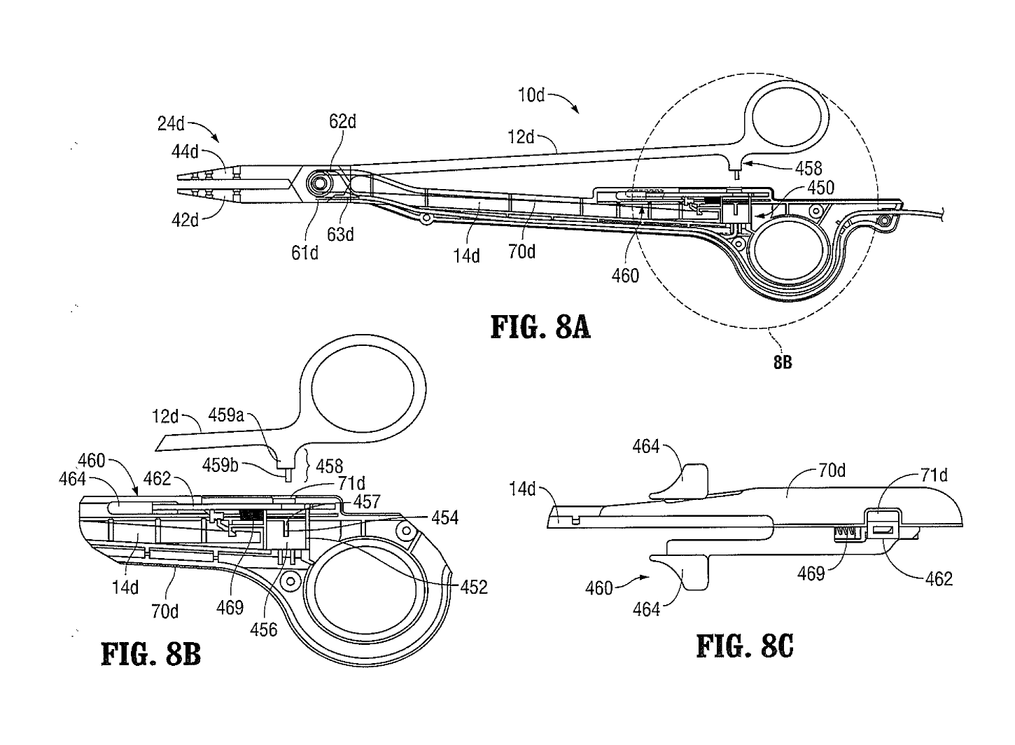

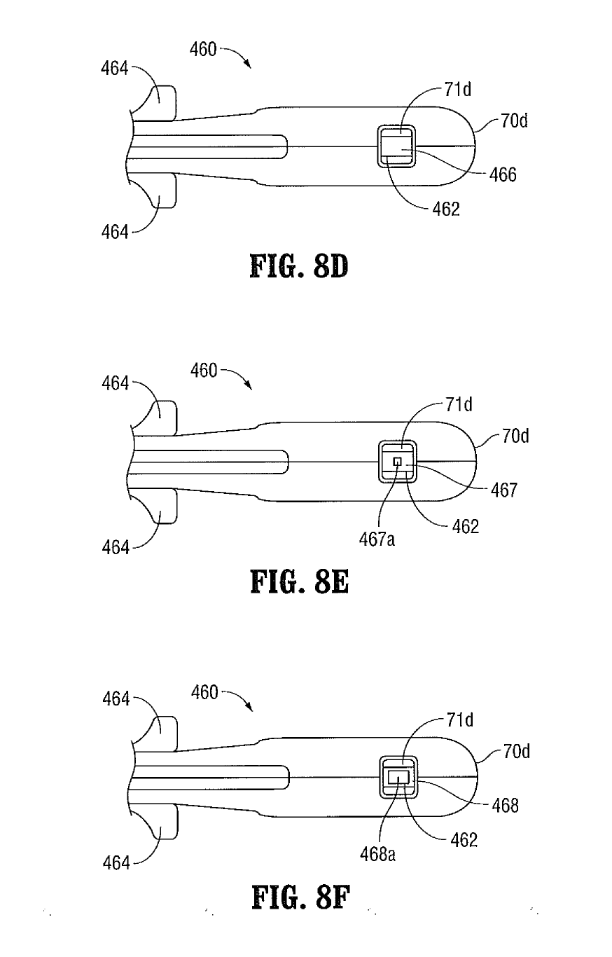

Turning now to FIGS. 8A-8F, another forceps 10d similar to forceps 10 (FIGS. 1-3) is shown including a two-mode switch assembly 450 and a safety selector 460. Two-mode switch assembly 450 is coupled between the source of energy (not shown) and end effector assembly 24d via wires 61d, 62d, 63d. As such, two-mode switch assembly 450 is configured for activation in a first mode for energizing electrodes, e.g., electrodes 110, 120 (FIG. 3), for operation in a tissue treatment mode, e.g., for sealing tissue grasped between jaw members 42d, 44d, and in a second mode for energizing electrodes, e.g., electrodes 110, 120, 130 (FIG. 3), for operation in an electrical cutting mode, e.g., for cutting tissue grasped between jaw members 42d, 44d. Safety selector 460 is selectively movable between a first position (FIG. 8D), wherein activation of two-mode switch assembly 450 in both the first and second modes is inhibited; a second position (FIG. 8E), wherein activation of two-mode switch assembly 450 in the first mode is permitted but activation in the second mode is inhibited; and a third position (FIG. 8F), wherein activation of two-mode switch assembly 450 in both the first and second modes is permitted.

Two-mode switch assembly 450 is seated within a recess defined within housing 70d and is accessible via a window 71d defined within housing 70d, e.g., defined partly by each housing portion of housing 70d. Two-mode switch assembly 450 includes a sleeve 452 fixedly engaged within housing 70d and inner and outer buttons 454, 456, respectively, disposed within sleeve 452. Inner and outer buttons 454, 456 are depressible relative to sleeve 452 to activate two-mode switch assembly 450 in the first and second modes, respectively. More specifically, outer button 456 defines an aperture 457 through which inner button 454 extends, thus permitting independent actuation of inner button 454.

Shaft member 12d includes a tiered engagement flange 458 extending therefrom towards two-mode switch assembly 450. More specifically, tiered engagement flange 458 includes a base portion 459a defining a relatively large width and an extension portion 459b defining a relatively narrower width, centered on base portion 459a, and extending from base portion 459a towards two-mode switch assembly 450. Upon sufficient approximation of shaft members 12d, 14d, extension portion 459b is inserted into aperture 457 of outer button 456 to depress and activate inner button 454 without the need for activation of outer button 456. Thus, activation of two-mode switch assembly 450 in only the first mode is possible. Base portion 459a, on the other hand, is dimensioned larger than aperture 457 such that, upon further approximation of shaft members 12d, 14d, base portion 459a contacts outer button 456 to depress and activate outer button 456. In this situation, where both inner and outer buttons 454, 456 are depressed, two-mode switch assembly 450 is activated in the second mode.

By requiring further approximation of shaft members 12d, 14d to activate two-mode switch assembly 450 in the second mode, e.g., for electrically cutting tissue, as compared to the first mode, e.g., for tissue sealing, jaw members 42d, 44d are further approximated during tissue cutting as compared to tissue sealing. Such a feature is advantageous in that a larger clamping pressure on tissue is desirable in order to effect electrical tissue cutting as compared to tissue sealing.

As mentioned above, two-mode switch assembly 450 is configured for activation in a first mode or a second mode depending on the degree of approximation of shaft members 12d, 14d. As also mentioned above, safety selector 460 is selectively movable between a first position (FIG. 8D), a second position (FIG. 8E), and a third position (FIG. 8F) for selectively inhibiting activation of two-mode switch assembly 450 in either or both of the first and second modes. Alternatively, safety selector 460 may be configured to include only two positions, e.g., the first and third positions or the second and third positions, depending on a particular purpose. The configuration and operation of safety selector 460 is described below.

Referring to FIGS. 8D-8F, in conjunction with FIGS. 8A-8C, safety selector 460 generally includes a control member 462 and a pair of grasping flanges 464. Grasping flanges 464 are disposed on either side of housing 70d to facilitate operation of safety selector 460 from either side of forceps 10d. Grasping flanges 464 extend through slots defined within housing 70d to engage control member 462 at the distal end of control member 462. Control member 462 extends proximally through housing from the distal end thereof to the proximal end thereof. The proximal end of control member 462 includes first, second, and third segments 466, 467, 468, respectively, that are configured for positioning within window 71d defined within housing 70d in the respective first, second, and third positions (FIGS. 8D, 8E, and 8F, respectively) of safety selector 460, as will be described in greater detail below. Grasping flanges 464 are movable along housing 70d to translate control member 462 between the first, second, and third positions (FIGS. 8D, 8E, and 8F, respectively). A biasing member 469 biases control member 462 towards the first position. Further, any suitable releasable latching mechanism(s) or releasable engagement structure(s) (not shown) may be provided for releasably retaining control member 462 in the second and/or third position.

As shown in FIG. 8D, in conjunction with FIGS. 8A-8C, first segment 466 of control member 462 defines a solid, uninterrupted configuration, thereby inhibiting passage of both base portion 459a and extension portion 459b of flange 458 therethrough. As such, in the first position of control member 462, activation of two-mode switch assembly 450 in either mode of operation is inhibited.

As shown in FIG. 8E, in conjunction with FIGS. 8A-8C, second segment 467 of control member 462 defines a relatively small sized aperture 467a configured to permit passage of extension portion 459b of flange 458 therethrough but to inhibit passage of base portion 459a of flange 458 therethrough. As such, in the second position of control member 462, activation of two-mode switch assembly 450 in only the first mode of operation is permitted.

As shown in FIG. 8F, in conjunction with FIGS. 8A-8C, third segment 468 of control member 462 defines a relatively large sized aperture 468a configured to permit passage of both extension portion 459b of flange 458 and base portion 459a of flange 458 therethrough. As such, in the third position of control member 462, activation of two-mode switch assembly 450 in either mode of operation is permitted.

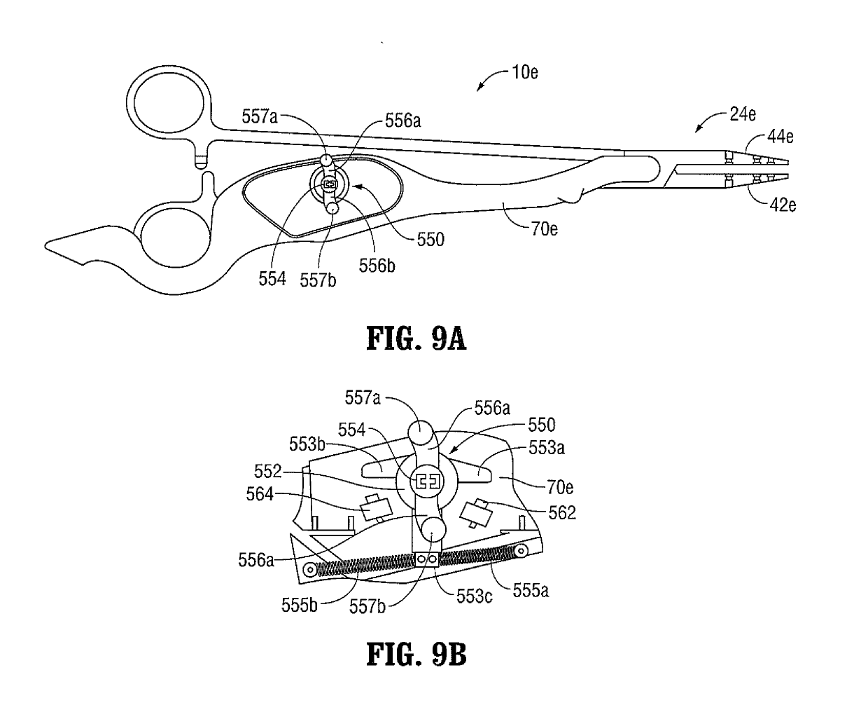

Turning now to FIGS. 9A and 9B, another forceps 10e similar to forceps 10 (FIGS. 1-3) is shown including a two-mode rotating switch assembly 550 disposed on either side of forceps 10e (although only one side is shown and referred to herein for purposes of simplicity). Two-mode rotating switch assembly 550 is configured for activation in a first mode for energizing electrodes, e.g., electrodes 110, 120 (FIG. 3), for operation in a tissue treatment mode, e.g., for sealing tissue grasped between jaw members 42e, 44e, and in a second mode for energizing electrodes, e.g., electrodes 110, 120, 130 (FIG. 3), for operation in an electrical cutting mode, e.g., for cutting tissue grasped between jaw members 42e, 44e. Two-mode rotating switch assembly 550 is coupled between the source of energy (not shown) and end effector assembly 24e similarly as described above with respect to previous embodiments.

Two-mode rotating switch assembly 550 is mounted on housing 70e of forceps 10e and includes inner and outer rotating members 552, 554, respectively, and first and second activation buttons 562, 564, respectively. Inner and outer rotating members 552, 554 are engaged with one another such that rotation of outer rotating member 554 effects corresponding rotation of inner rotating member 552. Inner rotating member 552 is disposed within housing 70e of and includes first, second, and third flanges 553a, 553b, 553c, respectively, extending radially outwardly from inner rotating member 552. First and second flanges 553a, 553b generally oppose one another, the use of which will be described in greater detail below. Third flange 553c is coupled to a pair of opposing biasing members 555a, 555b configured to bias two-mode rotating switch assembly 550 toward a neutral position.

Outer rotating member 554 includes a pair of generally opposed grasping arms 556a, 556b, each including an outwardly-protruding nub 557a, 557b, respectively, disposed at the free end thereof to facilitate grasping and rotating arms 556a, 556b. As will be described in greater detail below, sufficient rotation of arms 556a, 556b from the neutral position in a first direction, e.g., a clockwise direction as viewed in FIGS. 9A and 9B, activates two-mode rotating switch assembly 550 for operation in the first mode, while sufficient rotation of arms 556a, 556b from the neutral position in a second, opposite direction, e.g., a counterclockwise direction as viewed in FIGS. 9A and 9B, activates two-mode rotating switch assembly 550 for operation in the second mode.

First and second activation buttons 562, 564 of two-mode rotating switch assembly 550 are mounted within housing 70e and are positioned on either side of inner rotating member 552. More specifically, first activation button 562 is oriented to face and is positioned within the rotation path of first flange 553a of inner rotating member 552, while second activation button 564 is oriented to face and is positioned within the rotation path of second flange 553b of inner rotating member 552. First activation button 562 is coupled between the source of energy and the electrodes, e.g., electrodes 110, 120 (FIG. 3), such that, upon activation of first activation button 562, electrode 110 (FIG. 3) is energized to a relative positive potential and electrode 120 (FIG. 3) to a relatively negative potential (although this configuration may be reversed) to establish a potential gradient between electrodes 110, 120 (FIG. 3) for conducting energy through tissue grasped between jaw members 42e, 44e to treat, e.g., seal, tissue. As such, grasping and rotating either or both arms 556a, 556b in the first direction rotates first flange 553a into contact with first activation button 562 to activate first activation button 562 for tissue treatment, e.g., tissue sealing.

Second activation button 564 is coupled between the source of energy and the electrodes, e.g., electrodes 110, 120, 130 (FIG. 3), such that, upon activation of second activation button 564, electrical cutting electrode 130 is energized to a relatively positive potential and electrodes 110, 120 (FIG. 3) to a relatively negative potential for conducting energy through tissue grasped between jaw members 42e, 44e to cut tissue. As such, grasping and rotating either or both of arms 556a, 556b in the second direction rotates second flange 553b into contact with second activation button 564 to activate second activation button 562 for tissue cutting.

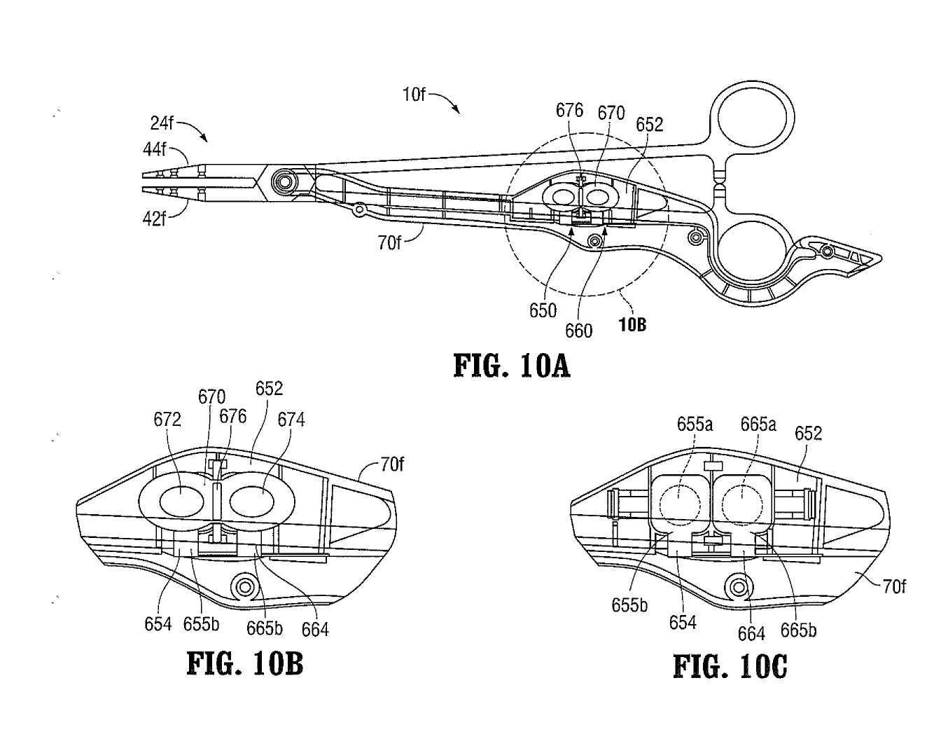

Turning now to FIGS. 10A-10C, another forceps 10f similar to forceps 10 (FIGS. 1-3) is shown including an activation first and second switch assemblies 650, 660 disposed within housing 70f and coupled between the source of energy (not shown) and end effector assembly 24f for selectively energizing electrodes 110, 120 (FIG. 3) for operation in a tissue treatment mode, e.g., for sealing tissue grasped between jaw members 42f, 44f, and for operation in a tissue cutting mode, e.g., for electrically cutting tissue grasped between jaw members 42f, 44f, respectively.

First and second switch assemblies 650, 660 are mounted on a frame, 652, similar to frame 152 (FIGS. 5A-5C) and each includes a flex circuit assembly 654, 664, respectively, similar to flex circuit assembly 156 (FIGS. 5A-5C), having a dome switch 655a, 665b disposed on each of the flanges 655b, 665b thereof. For purposes of brevity, only the differences between first and second switch assemblies 650, 660 and first switch assembly 150 (FIGS. 5A-5C) will be described in detail below.

First and second switch assemblies 650, 660 are selectively and alternatively activated via depressing lever member 670 in the vicinity of the desired switch assembly 650, 660 to be activated. More specifically, lever member 670 is mounted about a fulcrum 676 and defines a first end 672 disposed adjacent first switch assembly 650 and a second end 674 disposed adjacent second switch assembly 660. Lever member 670 is selectively and alternatively tiltable about fulcrum 676 towards first switch assembly 650, e.g., such that first end 672 of lever member 670 contact and urges dome switch 655a into an activated position, and towards second switch assembly 660, e.g., such that second end 674 of lever member 670 urges dome switch 665a into an activated position. Thus, upon sufficient tilting of lever member 670 to activate first switch assembly 650, tissue treatment, e.g., sealing, can be effected while, on the other hand, upon sufficient tilting of lever member 670 to activate second switch assembly 660, electrical tissue cutting can be effected.

While several embodiments of the disclosure have been shown in the drawings and described herein, it is not intended that the disclosure be limited thereto, as it is intended that the disclosure be as broad in scope as the art will allow and that the specification be read likewise. Therefore, the above description should not be construed as limiting, but merely as examples of particular embodiments. Those skilled in the art will envision other modifications within the scope and spirit of the claims appended hereto.

What is claimed is:

1. A surgical instrument, comprising:
an end effector assembly including first and second jaw members, at least one of the jaw members movable relative to the other to grasp tissue therebetween, at least one of the jaw members adapted to connect to a source of energy for treating tissue grasped between the jaw members, at least one of the jaw members adapted to connect to a source of energy for electrically cutting tissue grasped between the jaw members;
a first switch assembly operably coupled to the end effector assembly, the first switch assembly selectively activatable for supplying energy to the at least one jaw member for treating tissue grasped between the jaw members; and
a second switch assembly operably coupled to the end effector assembly, the second switch assembly selectively activatable for supplying energy to the at least one jaw member for electrically cutting tissue grasped between the jaw members, wherein the tactile feel and range of motion during actuation of the second switch assembly to effect electrical tissue cutting mimics the tactile feel and range of motion of activation of a mechanical actuator that advances a cutting blade between the jaw members to mechanically cut tissue.

2. The surgical instrument according to claim 1, wherein the first switch assembly includes a depressible button.

3. The surgical instrument according to claim 1, wherein the first switch assembly includes a flex circuit.

4. The surgical instrument according to claim 1, wherein the first switch assembly includes a dome switch.

5. The surgical instrument according to claim 1, wherein the first and second switch assemblies are coupled to a progressive switch.

6. The surgical instrument according to claim 1, wherein the second switch assembly includes a rotatable lever disposed on each side of the surgical instrument, each of the levers rotatable from a first position to a second position to activate the second switch assembly.

7. The surgical instrument according to claim 6, wherein the rotatable lever is biased towards the first position.

8. The surgical instrument according to claim 1, further comprising first and second shaft members operably coupled to the end effector assembly, the first and second shaft members movable relative to one another between a spaced-apart position and an approximated position for moving the jaw members relative to one another to grasp tissue therebetween.

9. The surgical instrument according to claim 8, wherein the first switch assembly is positioned such that movement of the first and second shaft members from the spaced-apart position to the approximated position activates the first switch assembly.

10. A surgical instrument, comprising:
an end effector assembly including first and second jaw members, at least one of the jaw members movable relative to the other to grasp tissue therebetween, at least one of the jaw members adapted to connect to a source of energy for treating tissue grasped between the jaw members, at least one of the jaw members adapted to connect to a source of energy for electrically cutting tissue grasped between the jaw members;
first and second shaft members coupled to the end effector assembly, the first and second shaft members movable relative to one another between a spaced-apart position and first and second approximated positions for moving the jaw members relative to one another between an open position and first and second grasping positions, the first shaft member including a flange extending therefrom towards the second shaft member, the flange including a first portion and a second portion; and
a two-mode switch assembly coupled to the second shaft member, the switch assembly including a first switch member selectively activatable for activating the switch assembly in a first mode for supplying energy to the at least one jaw member for treating tissue grasped between the jaw members, and a second switch member selectively activatable for activating the switch assembly in a second mode for supplying energy to the at least one jaw member for electrically cutting tissue grasped between the jaw members, wherein movement of the shaft members to the first approximated position urges the first portion of the flange into the first switch member to activate the first switch member, and wherein movement of the shaft members to the second approximated position urges the second portion of the flange into the second switch member to activate the second switch member.

11. The surgical instrument according to claim 10, wherein the two-mode switch assembly is disposed within a housing positioned about the second shaft member.

12. The surgical instrument according to claim 10, wherein the second portion of the flange defines a relatively wide base extending from the first shaft member and wherein the first portion of the flange defines a relatively narrow extension extending from the base.

13. The surgical instrument according to claim 12, wherein the first switch member of the two-mode switch assembly is disposed within an aperture defined through the second switch member.

14. The surgical instrument according to claim 10, further comprising a safety selector, the safety selector selectively movable between a first position, inhibiting activation of both the first and second switch members of the two-mode switch assembly, a second position inhibiting activation of the second switch member of the two-mode switch assembly but permitting activation of the first switch member of the two-mode switch assembly, and a third position permitting activation of both the first and second switch members of the two-mode switch assembly.

15. The surgical instrument according to claim 14, wherein the safety selector includes at least one gripping flange, the at least one gripping flange configured to facilitate movement of the safety selector between the first, second, and third positions.

16. The surgical instrument according to claim 14, wherein the safety selector is slidable along the second shaft member and relative to the two-mode switch assembly between the first, second, and third positions.

17. A surgical instrument, comprising:
an end effector assembly including first and second jaw members, at least one of the jaw members movable relative to the other to grasp tissue therebetween, at least one of the jaw members adapted to connect to a source of energy for treating tissue grasped between the jaw members, at least one of the jaw members adapted to connect to a source of energy for electrically cutting tissue grasped between the jaw members;
a first switch member selectively activatable for supplying energy to the at least one jaw member for treating tissue grasped between the jaw members;
a second switch member selectively activatable for supplying energy to the at least one jaw member for electrically cutting tissue grasped between the jaw members; and
an activation member including first and second activation components, the activation member movable in a first direction for urging the first activation component into the first switch member for activating the first switch member and movable in a second direction opposite the first direction for urging the second activation component into the second switch member for activating the second switch member.

18. The surgical instrument according to claim 17, wherein the activation member includes a rotating assembly having first and second flanges, the rotating assembly rotatable in the first direction such that the first flange is urged into contact with the first switch member to activate the first switch member and rotatable in the second direction such that the second flange is urged into contact with the second switch member to activate the second switch member.

19. The surgical instrument according to claim 18, wherein the rotating assembly is biased towards a neutral position wherein both the first and second flanges are displaced from the first and second switch members, respectively.

20. The surgical instrument according to claim 17, wherein the activation member includes a lever disposed about a fulcrum, the lever including a first end and a second end, the lever tiltable about the fulcrum in the first direction such that the first end is urged into contact with the first switch member to activate the first switch member and tiltable in the second direction such that the second end is urged into contact with the second switch member to activate the second switch member.
